(19) United States
(12) Patent Application Publication
(10) Pub. No.: US 2022/0193438 A1
(43) Pub. Date: Jun. 23, 2022

(54) BEAUTY CARE METHOD OF ENHANCING SKIN DEFENSE FUNCTION, METHOD OF FACILITATING EXPRESSION OF ANTIBACTERIAL PEPTIDE, METHOD OF FACILITATING EXPRESSION OF SIRTUIN 1, AND APPARATUS FOR FACILITATING EXPRESSION OF SIRTUIN 1

(71) Applicant: Bloom Classic Co., Ltd., Tokyo (JP)

(72) Inventors: Hayami Tabe, Tokyo (JP); Akihito Fujita, Tokyo (JP); Mayumi Kotani, Toyko (JP); Hidemi Sugiwaki, Toyko (JP)

(21) Appl. No.: 17/493,136

(22) Filed: Oct. 4, 2021

(30) Foreign Application Priority Data
Dec. 23, 2020 (JP) 2020213785
Jul. 2, 2021 (JP) 2021110812

(51) Int. Cl. A61N 2/02; A61N 2/00

(52) U.S. Cl. CPC A61N 2/02; A61N 2/004

(57) ABSTRACT

Proposed is a beauty care method of enhancing a skin defense function, in which a wave at a Schumann resonance frequency is applied to a cell. A method of facilitating expression of an antibacterial peptide and a method of facilitating expression of sirtuin 1 are also proposed, in each of which a wave at a Schumann resonance frequency is applied to a cell. Furthermore, an apparatus for facilitating expression of an antibacterial peptide and an apparatus for facilitating expression of sirtuin 1 are proposed, each of which includes an electromagnetic wave generation unit configured to generate a wave at a Schumann resonance frequency, and in each of which the wave at the Schumann resonance frequency is applied to a cell.

### SEQUENCE LISTING

The Sequence Listing file entitled “sequencelisting” having a size of 1,648 bytes and a creation date of Oct. 1, 2021 that was filed with the patent application is incorporated herein by reference in its entirety.

### TECHNICAL FIELD

The present invention relates to a beauty care method capable of enhancing a skin defense function. More particularly, the present invention relates to a method of facilitating expression of an antibacterial peptide, a method of facilitating expression of sirtuin 1, an apparatus for facilitating expression of an antibacterial peptide, and an apparatus for facilitating expression of sirtuin 1.

### BACKGROUND ART

Stresses, such as internal and external stimuli and aging, impede activating the innate immunity and adaptive immunity of the skin, particularly, the epidermis. Thus, the skin, particularly, the epidermis, cannot maintain water and pH indicating slight acidity, and a change occurs in a resident bacterial layer of the skin, thereby causing inflammation and infection. When a skin defense function works in a less effective manner, skin diseases are caused, such as atopic dermatitis, psoriasis, contact dermatitis, hypertrophic scarring, and common warts, and a thermal injury is not healed.

An antibacterial peptide plays an essential role in the innate immunity of the skin. It is known that defensin and cathelicidin (LL-37) are expressed in mammalian skin. It is known that types of defensin include α-defensin, β-defensin, and the like. Furthermore, cathelicidin is cleaved by a protease, and a C-terminal 37 amino acid residue is separated and acts as an antibacterial peptide LL-37.

Expression of defensin and expression of cathelicidin (LL-37) are facilitated in an epidermal keratinocyte due to skin damage and inflammation. Defensin and cathelicidin (LL-37) exhibit an antibacterial activity against pathogens, such as bacteria, fungi, and viruses (Non-patent Document 1). It is known that these antibacterial peptides have not only a bacteria destroying function, but also many biological defense functions, such as an immuno-regulation function, an anti-inflammation function, and a wound healing function (a function of facilitating neovascularization, cell migration, and cell growth and thus quickly healing a wound) (Non-patent Document 2).

Sirtuin 1 has NAD-dependent deacetylation enzyme activity and ADP ribosyl transferase activity and plays an important role within a living body.

For example, it is disclosed in Non-patent Document 3 that an experiment on mice that lost epidermis-specific sirtuin 1 showed that sirtuin 1 of the epidermis regulated cell migration, wound-induced innate immunity, epidermis re-epithelialization, granulation tissue formation, and neovascularization and was necessary for efficient wound healing (Non-patent Document 3).

In addition, improvements in carbohydrate metabolism, cholesterol metabolism, and fat metabolism, as well as an improvement in the physical ability and an extension of a reproductive duration were recognized in the mice in which high expression of sirtuin 1 was observed. Furthermore, an improvement in glucose tolerance and fatty liver inhibition were also observed after a high-fat meal. In summary, it is considered that activation of sirtuin 1 is effective in preventing or healing a disease associated with a metabolic system or alleviating the disease (Non-patent Document 4).

Therefore, it is considered that facilitation of expression of the antibacterial peptide and expression of sirtuin 1 can enhance skin defense functions.

MicroRNA (miRNA) is a single-stranded short-chain RNA having a length of approximately 20 to 25 bases and is a non-coding RNA that is not translated into a protein. The miRNA regulates expression of a gene and plays an important role in a molecular pathology of a disease, as well as in cellular creation and differentiation processes.

For example, it is reported that expression of a sirtuin 1 gene is reduced in a human epidermal keratinocyte into which miR-181a and miR-181b associated with aging and cell growth are introduced (Non-patent Document 5). Furthermore, it is reported that expression of miR-132 is increased in human epithelium cells processed with a transforming growth factor (TGF-β1 or TGF-β2) that is a cell growth inhibition factor (Non-patent Document 6). In addition, it is reported that expression of miR-145 is reduced in a legion portion of a patient with psoriasis disease (Non-patent Document 7) and that expression of miR-4654 is reduced in a fibroblast obtained from a legion portion where a hypertrophic scar is formed (Non-patent Document 8). It is reported that expression of miR-647 is increased in the skin infected with common warts (Non-patent Document 9) and that expression of miR-1973 is increased in an epidermal stem cell where thermal injury occurs (at a temperature of 51.5° C. for 35 seconds) (Non-patent Document 10).

It is considered that in this manner, miRNA is involved in facilitation of the expression of sirtuin 1, cell growth, and various skin diseases and that the facilitation or inhibition of the expression of these miRNAs can enhance the skin defense function.

It is disclosed in Patent Document 1 that the facilitation of the expression of the antibacterial peptide derived from the human skin, an active ingredient of which is a butcher bloom extract can enhance a skin barrier function or can achieve the prevention or alleviation of atopic dermatitis.

### DOCUMENTS OF RELATED ART

### Patent Document

(Patent Document 1): Japanese Patent Application Publication No. 2018-104364

### Non-Patent Documents

(Non-patent Document 1): N Engl J Med, 347: 1151-1160, 2002
(Non-patent Document 2): Chemistry and Biology, 57: 296-303, 2019
(Non-patent Document 3): Sci Rep, Article number 14110, 2017
(Non-patent Document 4): Chemistry and Biology, 47: pp. 531-537, 2009
(Non-patent Document 5): PNAS, 109: 1133-1138, 2012
(Non-patent Document 6): J Clin Invest, 125: 3008-3026, 2015
(Non-patent Document 7): Br J Dermatol, 180: 365-372, 2019
(Non-patent Document 8): Mol Med Rep, 22: 3440-3452, 2020
(Non-patent Document 9): Biomolecules, 9: 1-12, 2019
(Non-patent Document 10): Exp Ther Med, 19: 2218-2228, 2020

### DISCLOSURE

### Technical Problem

An objective of the present invention is to provide a beauty care method capable of enhancing a skin defense function.

Another objective of the present invention is to provide a method of facilitating expression of an antibacterial peptide and an apparatus for facilitating expression of an antibacterial peptide.

Still another objective of the present invention is to provide a method of facilitating expression of sirtuin 1 and an apparatus for facilitating expression of sirtuin 1.

### Technical Solution

According to an aspect of the present invention, there is provided a beauty care method of enhancing a skin defense function, in which a wave at a Schumann resonance frequency is applied to a cell.

According to another aspect of the present invention, there are provided a method of facilitating expression of an antibacterial peptide and a method of facilitating expression of sirtuin 1, in each of which a wave at a Schumann resonance frequency is applied to a cell. According to still another aspect of the present invention, there are provided an apparatus for facilitating expression of an antibacterial peptide and an apparatus for facilitating expression of sirtuin 1, each of which includes an electromagnetic wave generation unit configured to generate a wave at a Schumann resonance frequency, and in each of which the wave at the Schumann resonance frequency is applied to a cell.

### Advantageous Effects

With the beauty care method of enhancing a skin defense function according to the present invention, a skin defense function can be enhanced by applying the wave at the Schumann resonance frequency to the cell.

With the method of facilitating expression of an antibacterial peptide and the apparatus for facilitating expression of an antibacterial peptide according to the present invention, the expression of the antibacterial peptide can be facilitated by applying the wave at the Schumann resonance frequency to the cell.

With the method of facilitating expression of sirtuin 1 and the apparatus for facilitating expression of sirtuin 1, the expression of sirtuin 1 can be facilitated by applying the wave at the Schumann resonance frequency to the cell.

### DESCRIPTION OF DRAWINGS

FIG. 1 is photographs each showing an operation of migrating a cell by applying a wave at a Schumann resonance frequency to the cell. It is noted that the bar on a lower portion of each of the photographs has a length of 0.6 μm.

FIG. 2 is a table showing absorbance of 540 nm for comparative and implementation examples.

### MODE FOR INVENTION

Embodiments of the present invention will be described below.

[Beauty Care Method, a Method of Facilitating Expression of an Antibacterial Peptide, and a Method of Facilitating Expression of Sirtuin 1]

In a beauty care method of enhancing a skin defense function (hereinafter referred to as a “beauty care method” for short) according to the present embodiment, a wave at a Schumann resonance frequency is applied to a cell.

In a method of facilitating expression of an antibacterial peptide according to the present embodiment, the wave at the Schumann resonance frequency is applied to the cell. Accordingly, expression of an antibacterial peptide can be facilitated, and the skin defense function can be enhanced. Furthermore, in a method of facilitating expression of sirtuin 1 according to the present embodiment, the wave at the Schumann resonance frequency is applied to the cell. Accordingly, the expression of sirtuin 1 can be facilitated, and the skin defense function can be enhanced.

The Schumann resonance is an electromagnetic resonance phenomenon where resonance and sympathetic vibration take place with the limited dimensions of the earth, and are stationary over the surface of the earth. Schumann resonance frequencies are in the following order starting from the lowest: approximately 7.8 Hz, 14.1 Hz, 20.3 Hz, 26.4 Hz, and 32.4 Hz. The Schumann resonance frequencies of the wave that are applied to the cell according to the present embodiment may range from 7.7 to 7.9 Hz, from 13.9 to 14.3 Hz, from 20.0 to 20.6 Hz, and from 26.0 to 26.8 Hz. According to the present embodiment, the Schumann resonance frequency of the wave that is applied to the cell may range from 7.7 to 7.9 Hz, from 13.9 to 14.3 Hz, from 20.0 to 20.6 Hz, and from 26.0 to 26.8 Hz.

In the beauty care method, the method of facilitating expression of an antibacterial peptide, and the method of facilitating expression of sirtuin 1 according to the present embodiment, it is preferred that the Schumann resonance frequency of the wave that is applied to the cell ranges from 7.7 to 7.9 Hz and from 13.9 to 14.3 Hz, particularly, from 7.7 to 7.9 Hz.

In the beauty care method, the method of facilitating expression of an antibacterial peptide, and the method of facilitating expression of sirtuin 1 according to the present embodiment, a cell to which the wave at the Schumann resonance frequency can be applied is not particularly limited. An epidermal cell, particularly, an epidermal keratinocyte is preferred.

The beauty care method, the method of facilitating expression of an antibacterial peptide, and the method of facilitating expression of sirtuin 1 according to the present embodiment can be implemented, for example, using an apparatus or the like that generates the wave at the Schumann resonance frequency. Specifically, such implementation is made possible by applying the wave at the Schumann resonance frequency generated from the apparatus to the cell for a predetermined amount of time.

In the beauty care method, the method of facilitating expression of an antibacterial peptide, and the method of facilitating expression of sirtuin 1 according to the present embodiment, the wave at the Schumann resonance frequency is successively applied, preferably for 1 to 30 minutes, and more preferably for 5 to 20 minutes. Furthermore, in the case of the application for 5 to 10 minutes per day, it is preferred that such application takes place successively for 1 to 7 days.

Beauty care by the application of the wave at the Schumann resonance frequency takes effect on the basis of an operation of enhancing the skin defense function. At this point, the operation of enhancing the skin defense function, for example, is achieved on the basis of one or more operations selected from among an operation of facilitating expression of an antibacterial peptide, an operation of facilitating expression of sirtuin 1, an operation of migrating a cell, a cell growth operation, an operation of inhibiting expression of miR-181a, an operation of inhibiting expression of miR-181b, an operation of inhibiting expression of miR-132, an operation of facilitating expression of miR-145, an operation of facilitating expression of miR-4654, an operation of inhibiting expression of miR-647, and an operation of inhibiting expression of miR-1973. However, the operation of enhancing the skin defense function is not limited to the operation of enhancing the skin defense function, which is based on the above operations.

The application of the wave at the Schumann resonance frequency to the cell makes the beauty care method according to the present embodiment applicable to the following intended applications through the operation of enhancing the skin defense function.

The application of the wave at the Schumann resonance frequency to the cell can enhance skin defense functions, such as a bacteria destroying function, an immuno-regulation function, an anti-inflammation function, and a wound healing function, through the operation of facilitating expression of an antibacterial peptide.

The application of the wave at the Schumann resonance frequency to the cell can enhance the skin defense functions, such as a cell migration function, a wound-induced innate immunity function, an epidermis re-epithelization function, a granulation tissue formation function, a neovascularization function, and a wound healing function, through the operation of facilitating expression of sirtuin 1.

Moreover, the application of the wave at the Schumann resonance frequency to the cell can enhance the wound healing function through the operation of migrating a cell and/or the cell growth operation.

The application of the wave at the Schumann resonance frequency to the cell can facilitate the expression of sirtuin 1 and can enhance the skin defense functions, such as the cell migration function, the wound-induced innate immunity function, the epidermis re-epithelization function, the granulation tissue formation function, the neovascularization function, and the wound healing function, through the operation of inhibiting expression of miR-181a and the operation of inhibiting expression of miR-181b.

The application of the wave at the Schumann resonance frequency to the cell can enhance a cell growth function through the operation of inhibiting expression of miR-132.

The application of the wave at the Schumann resonance frequency to the cell can enhance a function of healing inflammatory skin diseases, such as psoriasis, hypertrophic scarring, and common warts, and thermal injury, through the operation of facilitating expression of miR-145, the operation of facilitating expression of miR-4654, the operation of inhibiting expression of miR-647, and the operation of inhibiting expression of miR-1973.

Furthermore, the method of facilitating expression of an antibacterial peptide according to the present embodiment can be applied to the above-described intended applications based on the operation of facilitating expression of an antibacterial peptide. Moreover, the method of facilitating expression of sirtuin 1 according to the present embodiment can be applied to the above-described intended applications based on the operation of facilitating expression of sirtuin 1.

It is noted that according to the present embodiment, the inhibition of the expression of each of miR-181a, miR-181b, miR-132, miR-647, and miR-1973 and the facilitation of the expression of each of miR-145 and miR-4654 are made possible, thereby providing methods of modulating the expression of these miRNAs. That is, embodiments of the present invention are as follows.
Method of inhibiting expression of one or more types of miRNAs selected from a group consisting of miR-181a, miR-181b, miR-132, miR-647, and miR-1973, in which a wave at a Schumann resonance frequency is applied to a cell
Method of facilitating expression of miR-145 and/or miR-4654, in which a wave at a Schumann resonance frequency is applied to a cell

The method of modulating expression of each of these miRNAs can be applied to the above-described intended applications based on the operation of facilitating or inhibiting the expression of each miRNA.

At this point, miRNAs the expression of each of which is modulated according to the present embodiment are as shown in Table 1. It is noted that miRBase in Table 1 is a primary on-line database on miRNAs (http://www.mirabase.org) that is managed by the University of Manchester in the UK.

TABLE 1

| | | miRBase | | |
|---|---|---|---|---|
| | miRBase | accession | | Arrangement |
| miRNA | ID | number | Arrangement | number |
| miR- | Hsa- | MIMAT00 | accacugacc | Arrangement |
| 181a | miR- | 04558 | guugacugua | number 1 |
| | 181a- | | c | |
| | 2-3p | | | |
| miR- | Hsa- | MIMAT00 | cucacugauc | Arrangement |
| 181b | miR- | 31893 | aaugaaugca | number 2 |
| | 181b- | | | |
| | 2-3p | | | |
| miR- | Hsa- | MIMAT00 | uaacagucua | Arrangement |
| 132 | miR- | 00426 | cagccauggu | number 3 |
| | 132- | | c | |
| | 3p | | | |
| miR- | Hsa- | MIMAT00 | guggcugcac | Arrangement |
| 647 | miR- | 003317 | ucacuuccuu | number 4 |
| | 647 | | c | |
| miR- | Hsa- | MIMAT00 | accgugcaaa | Arrangement |
| 1973 | miR- | 09448 | gguagcaua | number 5 |
| | 1973 | | | |
| miR- | Hsa- | MIMAT00 | guccaguuuu | Arrangement |
| 145 | miR- | 00437 | cccaggaauc | number 6 |
| | 145- | | c | |
| | 5p | | | |
| miR- | Has- | MIMAT00 | ugugggaucu | Arrangement |
| 4654 | miR- | 19720 | ggaggcaucu | number 7 |
| | 4654 | | g | |

Examples of the skin subject to the enhancement of the skin defense function and/or the facilitation of the wound healing function include skins infected with skin diseases, such as atopic dermatitis, psoriasis, contact dermatitis, hypertrophic scarring, and common warts, a skin on which thermal injury is inflicted, a skin whose skin defense function works in a less effective manner due to various stresses, skin roughness, or the like, a skin whose skin defense function is degenerated due to transplantation or the like, a skin where a wound occurs, and the like.

Although there is no particularly limited antibacterial peptide, defensin and/or cathelicidin are preferred as antibacterial peptides. Among types of defensin, β-defensin, particularly, β-defensin2 is preferred. Furthermore, among types of cathelicidin, LL-37 is preferred. It is noted that LL-37 results from cleaving a C-terminal 37 amino acid residue from a human cationic antibacterial polypeptide of 18-kDa (hCAP 18) that is a cathelicidin produced from an epidermal cell.

It is noted that the above-described methods according to the present embodiment may be excluded from being employed for a human medical practice and may be excluded from being employed for a human or animal medical practice.

[Apparatus for Facilitating Expression of an Antibacterial Peptide and an Apparatus for Facilitating Expression of Sirtuin 1]

In an apparatus for facilitating expression of an antibacterial peptide and an apparatus for facilitating expression of sirtuin 1 according to the present invention, a wave at a Schumann resonance frequency is applied to a cell. The method of facilitating expression of an antibacterial peptide or the method of facilitating expression of sirtuin 1 according to the present invention may be implemented using the apparatus for facilitating expression of an antibacterial peptide and the apparatus for facilitating expression of sirtuin 1, respectively. It is noted that time for use and a living body portion for use are as described above.

The apparatus for facilitating expression of an antibacterial peptide and the apparatus for facilitating expression of sirtuin 1 according to the present embodiment each include an electromagnetic wave generation unit configured to generate a wave at a Schumann resonance frequency. Examples of the electromagnetic wave generation unit include a coil, an antenna, and the like. Each of the apparatuses according to the present embodiment may further include a frequency adjustment unit adjusting a frequency.

The apparatus for facilitating expression of an antibacterial peptide according to the present embodiment can enhance the skin defense functions, such as the bacteria destroying function, the immuno-modulation function, the anti-inflammation function, and the wound healing function, through the operation of facilitating expression of an antibacterial peptide. Furthermore, the apparatus for facilitating expression of sirtuin 1 according to the present embodiment can enhance the skin defense functions, such as the cell migration function, the wound-induced innate immunity function, the epidermis re-epithelization function, the granulation tissue formation function, the neovascularization function, and the wound healing function through the operation of facilitating expression of sirtuin 1.

### EXAMPLES

The present invention will be described in detail below with reference to implementation examples and is not at all limited to each of the following implementation examples.

### Experimental Example 1: Experiment for the Operation of Migrating a Cell

Normal Human Epidermal Keratinocytes (NHEKs) derived from a child as a single donor (the number of successive subcultures: 2 to 4, manufactured by PromoCell GmbH) were cultured at a temperature of 37° C. in the presence of 5% CO₂, using Keratinocyte Growth Medium2 (KGM2, manufactured by PromoCell GmbH). After cultured, 2 mL of NHEK was placed in each 35 mm dish at a concentration of 12×10⁴ cells/mL. NHEK was cultured until a confluent state was entered and then was further cultured for 24 hours (G0 period). Cross-lines were drawn in the 35 mm dish using a 200 μL pipette tip, and thus a cell was peeled off. Then, NHEK was cleaned with a culture medium (KGM2) and was further cultured for 24 hours. This case was defined as Comparative Example 1 (n=3).

Furthermore, when peeling off the cell and after 24 hours of culturing, NHEK was cultured in the same manner as in Comparative Example 1 (n=3), except that NHEK was left unattended for 10 minutes on a generator generating a pulse at an extremely low frequency of 7.83 Hz (product name: “CF-FM783-BA”, and operating current: 1 mV, manufactured by Walfront LLC) and that an electromagnetic wave was then applied. This case was defined as Implementation Example 1.

Moreover, after peeling off of the cell, NHEK was cultured in the same manner as in Comparative Example 1 (n=3), except that NHEK was cultured for 24 hours using a culture medium (KGM2) containing a final 0.1 μg/mL concentration of synthetic diacylated lipopeptide derived from mycoplasma (Fibroblast-Stimulating Lipopeptide-1 (FSL-1), manufactured by Adipogen Life Sciences Inc.). It is known that the diacylated lipopeptide is recognized by a Toll-like receptor (TLR) and thus facilitates production of the antibacterial peptide. This case was defined as Comparative Example 2.

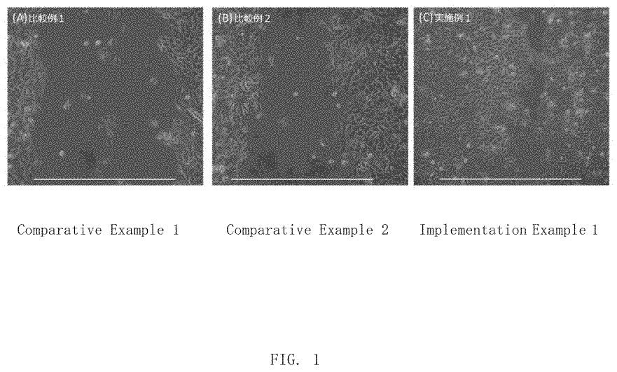

After 24 hours of culturing, a state of the vicinity of a groove created by reeling off the cell with a pipette tip was observed with a microscope in Implementation Example 1 and Comparative Examples 1 and 2. Photographs obtained by a microscope are shown in FIG. 1.

The excellent operation of migrating a cell was recognized in Implementation Example 1 (C) where an electromagnetic wave of 7.83 Hz (the wave of the Schuman resonance frequency) was applied than in Comparative Example 1(A) and Comparative Example 2(B).

### Experimental Example 2: An Experiment for an Operation of Facilitating Expression of β-Defensin 2 and Cathelicidin

After an experiment for the operation of migrating a cell was conducted in Experimental Example 1 (observation with a microscope), in each of Comparative Examples 1 and 2, the culture medium culture was removed, and cleaning was performed with a phosphate buffer solution. Subsequently, 1 mL of 1% sodium dodecyl sulfate (manufactured by Nippon Gene Co., Ltd.) was added and the cell was collected. Thus, this cell suspension solution was fully stirred by vortexing, and then 180 μL thereof was gathered. 1 μL of 1% KOH (manufactured by Nacalitesk Co., Ltd.) and 20 μL of 20 mg/mL Proteinase K Solution (manufactured by Thermo Fisher Scientific Inc.) were added to the cell suspension solution, and the result was incubated at a temperature of 37° C. for 15 minutes. After incubating, 100 μL of RNA Clean XP (manufactured by Beckman Coulten Inc.) was added, and the result was stirred and then was left at rest on a magnet for 5 minutes. Supernatant liquid was removed. The result was cleaned two times with 85% ethanol, and then was dried for 10 minutes. 30 μL of Nuclease-FreeWater (manufactured by Thermo Fisher Scientific Inc.) was added. The result was left at rest on a magnet for 5 minutes, and the supernatant was used as an RNA solution.

At a temperature of 0° C., in a 200 μL PCR tube (with Clear Dome Cap, manufactured by Bio-Rad Laboratories, Inc.), Nuclease-free water to Super Script IV VILO Master Mix were prepared at a ratio of 2.5 to 1, the result was stirred by vortexing, and then 14.0 μL was dispensed to each separate PCR tube. 6.0 μL of the RNA solution obtained as described above was added to this result. Reverse transcription reaction (at a temperature of 25° C. for 10 minutes, a temperature of 50° C. for 10 minutes, and a temperature of 85° C. for 5 minutes) was performed using a thermal cycler (product name: “T100 Thermal Cycler”, manufactured by Bio-Rad Laboratories, Inc.). Thus, a cDNA solution was obtained.

Taqman Gene Expression Assays (manufactured by Thermo Fisher Scientific Inc.) were used for cDNA amplification. Specifically, the same applied to cathelicidin (CAMP, Assay ID: Hs00189038_m1) and β-actin (ACTB, Assay ID: Hs99999903_m1) as endogenous control, using Taqman Gene Expression Assays (DEFB4A/DEFB4B, Assay ID: Hs00175474_m1) containing a primer and a probe that are capable of amplifying β-defensin2. TaqPath qPCR Master Mix, CG (manufactured by Thermo Fisher Scientific Inc.) and Nuclease-free water were prepared at a ratio of 10:5 with respect to Taqman Gene Expression Assays1. The result was placed in a nuclease-free tube, was stirred by vortexing and then was spun down. 6.0 μL of the result was dispensed to each PCR tube (with White Flat Cap, manufactured by Bio-Rad Laboratories, Inc.), and 4.0 μL of the cDNA solution (Implementation Example 1, and Comparative Example or Comparative Example 2) was added. The result was by pipetting and vortexing and then was spun down.

Using these samples, real-time PCR (product name: “C1000TouchThermal Cycler”, manufactured by Bio-Rad Laboratories, Inc.) was performed. The real-time PCR was performed in the following PCR conditions: at a temperature of 25° C. for 2 minutes, at a temperature of 95° C. for 20 seconds, at a temperature of 95° C. for 3 seconds (1), and at a temperature of 60° C. for 30 seconds (2) (40 cycles from (1) to (2)). An amount of expression each of B-defensin2 and cathelicidin was standardized with an amount of expression of β-activation. Expression facilitation ratios of β-defensin2 and cathelicidin were calculated as relative values with respect to an average value of amounts of expression of genes in Comparative Example 1 (non-processing) standardized, the average value being defined as 1. The results of the calculation are shown in Table 2.

TABLE 2

Expression facilitation ratio

| | β-defensin2 | cathelicidin |
|---|---|---|
| Comparative Example 1 | 1 | 1 |
| Comparative Example 2 | 13.76 | 1.79 |
| Implementation Example 1 | 26.32 | 34.00 |

From Table 2, it can be seen that the expression of each of β-defensin2 and cathelicidin was facilitated in Implementation Example 1 where an electromagnetic wave (the wave at the Schumann resonance frequency) of 7.83 Hz was applied to the cell, compared with Comparative Example 1 (non-processing). Furthermore, the expression of each o β-defensin2 and cathelicidin was also facilitated in Implementation Example 1, compared with Comparative Example 2 where a diacylated lipopeptide was added.

### Experimental Example 3: An Experiment for the Cell Growth Operation

Normal Human Epidermal Keratinocytes (NHEKs) derived from a child as a single donor (the number of successive subcultures: 2 to 4, manufactured by PromoCell GmbH) were cultured at a temperature of 37° C. in the presence of 5% CO₂, using Keratinocyte Growth Medium2 (KGM2, manufactured by PromoCell GmbH). After cultured, 100 μL of NHEK per well was placed in each of 96 well plates at a concentration of 4×10⁴ cells/mL and was cultured for 96 hours.

After 24 hours of culturing, 48 hours of culturing, and 72 hours of culturing, NHEK was left unattended for 10 minutes on the generator generating a pulse at an extremely low frequency of 7.83 Hz (product name: “CF-FM783-BA”, and operating current: 1 mV, manufactured by Walfront LLC) and then an electromagnetic wave was applied (n=6). This case was defined as Implementation Example 2. Furthermore, as control, non-processed wells were also prepared (n=6), and this case was defined as Comparative Example 3.

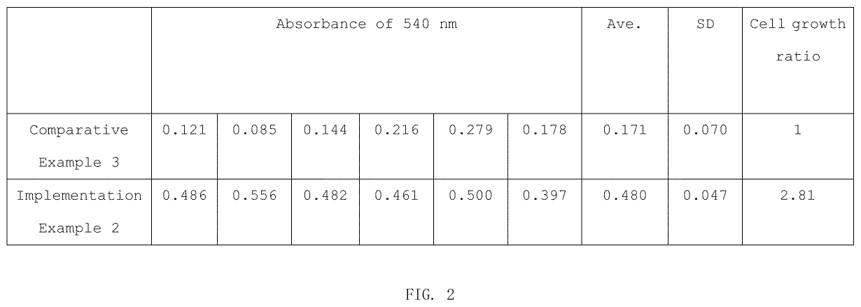

After 96 hours of culturing, a culture solution was removed, cleaning was performed using a phosphate buffer solution (manufactured by Sigma-Aldrich Japan Co Llc.), 100 μL of 50 μg/mL Neutral Red (NR) (manufactured by Nakalitesk company) was added to each well, and then culturing was performed for 3 hours. The NR liquid was removed, 200 μL of fixing liquid (1% formaldehyde) was added, and thus the cell was fixed. The fixing liquid was removed, 100 μL of extraction liquid (1% acetic acid, 50% 2-propanol) was added to each well, and NR entrapped in the cell was extracted. An absorbance of 540 nm was measured with a microplate reader (product name: “Immuno Mini NJ-2300”, manufactured by NaruGenk International Co.). Values of the absorbance of 540 nm are shown in the table of FIG. 2. A cell growth ratio was calculated as a relative value with respect to an average value of the absorbance of 540 nm in Comparative Example 3 (non-processing), the average value being defined as 1. The results of the calculation are shown in the table of FIG. 2.

From FIG. 2, it can be seen that the number of times that the cell growth operation takes place in Implementation 2 where an electromagnetic wave (the wave at the Schumann resonance frequency) of 7.83 Hz was applied to the cell is 2.81 times greater than the number of times that the cell growth operation takes place in Comparative Example 3 (non-processing).

### Experimental Example 4: An Experiment for the Operation of Facilitating Expression of Sirtuin 1

Normal Human Epidermal Keratinocytes (NHEK) derived from a child as a single donor (the number of successive subcultures: 2 to 4, manufactured by PromoCell GmbH) were cultured at a temperature of 37° C. in the presence of 5% CO₂, using Keratinocyte Growth Medium2 (KGM2, manufactured by PromoCell). After cultured, 5 mL of NHEK was placed in each T25 flask at a concentration of 3×10⁴ cells/mL (n=3 to 5). NHEK was further cultured for 72 hours. This case was defined as Comparative Example 4.

Furthermore, after placed, after 24 hours of culturing, 48 hours of culturing, and 72 hours of culturing, NHEK was left unattended for 10 minutes on the generator generating a pulse at an extremely low frequency of 7.83 Hz (product name: “CF-FM783-BA”, and operating current: 1 mV, manufactured by Walfront LLC) and then an electromagnetic wave of 7.83 Hz was applied. This case was defined as Implementation Example 3.

After 72 hours of culturing, in the same manner as in Experimental Example 2, in Implementation Example 3 and Comparative Example 4, the cell was collected, an RNA solution was obtained, the reverse transcription reaction was then performed, and a cDNA solution was obtained.

Taqman Gene Expression Assays (manufactured by Thermo Fisher Scientific Inc.) were used for cDNA amplification. Specifically, β-actin (ACTB, assay ID:Hs99999903_m1) was used as sirtuin 1 (SIRT1, assay ID:Hs01009006_m1) and endogenous control. Then, the real-time PCR was performed in the same manner as in Experimental Example 2.

An amount of expression of sirtuin 1 was standardized with an amount of expression of β-actin. An expression facilitation ratio of sirtuin 1 was calculated as a relative value with respect to an average value of amounts of expression of genes in Comparative Example 4 (non-processing) standardized, the average value being defined as 1. The results of the calculation are shown in Table 3.

TABLE 3

| | Expression facilitation ratio |
|---|---|
| | Sirtuin 1 |
| Comparative Example 4 | 1 |
| Implementation Example 3 | 4.9 |

From Table 3, it can be seen that the expression of sirtuin 1 was facilitated in Implementation Example 3 where an electromagnetic wave the wave at the Schumann resonance frequency) of 7.83 Hz was applied to the cell, compared with Comparative Example 4 (non-processing).

### Experimental Example 5: miRNA Analysis of an Exosome

5 mL of NHEK was placed in each T25 flask at a concentration of 3×10⁴ cells/ml (n=2). After 24 hours of culturing, 48 hours of culturing, and 72 hours of culturing, NHEK was left unattended for 10 minutes on the generator generating a pulse at an extremely low frequency of 7.83 Hz (product name: “CF-FM783-BA”, and operating current: 1 mV, manufactured by Walfront LLC) and then an electromagnetic wave was applied. This case was defined as Implementation Example 4. A case where an electromagnetic wave of 7.83 Hz was not applied was defined as Comparative Example 5 (non-processing). After 72 hours of culturing, a culture supernatant was provided to ExoQuick-TC (manufactured by System Bioscience LLC.), and thus an exosome was collected. The collected exosome (n=2) was suspended in 300 μL of PBS, and then was stored at a temperature −80° C. An RNA was extracted from the exosome, and then an amount of expression of miRNA was analyzed using an on-assignment analysis service provided by Toray Industries, Inc. In the on-assignment analysis service, labeling was performed with a 3D-Gene miRNA Labeling kit (manufactured by Toray Industries, Inc.), hybridization was performed on a 3D-Gene Human miRNA Oligo chip (ver.22, equipped with 2,632 types of miRNA detection probes, and manufactured by Toray Industries, Inc.) at a temperature of 32° C. for 16 hours, and then an image was acquired using a scanner (3D-Gene Scanner 3000, manufactured by Toray Industries, Inc.) and was analyzed using quantification software (3D-Gene Extraction software, manufactured by Toray Industries, Inc.).

A measurement value was corrected in such a manner that a median of values obtained as a result from measurement was 25 and was standardized with the 75-th percentile of the total 2,632 correction values. The result was defined as an amount of expression of each miRNA and a comparison was made between Implementation Example 4 and Comparative Example 5.

An amount of expression of each of miR-145-5p and miR-4654, which was at the same level as that of the background in Comparative Example 5, was remarkably facilitated in Implementation Example 4. When a correction value in Comparative Example 5 was defined as 1 for comparison in terms of an expression change ratio, the expression of miR-145-5p was calculated as being 12.19 times as high, and the expression of miR-4654 was calculated as being 12.40 times as high.

A significant amount of the expression of each of miR-181a-2-3p, miR-181b-2-3p, miR-132-3p, miR-647, and miR-1973 was detected in Comparative Example 5. However, in Implementation Example 4, the amount of the expression thereof was remarkably reduced to the same level as that of the background. When a correction value in Implementation Example 4 is defined as 1 for comparison in terms of the expression change ratio, the expression of miR-181a-2-3p was calculated as being 0.087 times as high, the expression of miR-181b-2-3p was calculated as being 0.116 times as high, the expression of miR-132-3p was calculated as being 0.090 times as high, the expression of miR-678 was calculated as being 0.089 times as high, and the expression of miR-1973 was calculated as being 0.091 times as high.

### INDUSTRIAL APPLICABILITY

With the beauty care method of enhancing a skin defense function according to the present invention can enhance the skin defense function. Furthermore, with the method for facilitating expression of an antibacterial peptide and the apparatus for facilitating expression of an antibacterial peptide, the expression of the antibacterial peptide can be facilitated. Moreover, with the method of facilitating expression of sirtuin 1 and the apparatus for facilitating expression of sirtuin 1, the expression of sirtuin 1 can be facilitated.

1. A beauty care method of enhancing a skin defense function, wherein a wave at a Schumann resonance frequency is applied to a cell.

2. A method of facilitating expression of an antibacterial peptide or facilitating expression of sirtuin 1, wherein a wave at a Schumann resonance frequency is applied to a cell.

3. An apparatus for facilitating expression of an antibacterial peptide or facilitating expression of sirtuin 1, the apparatus comprising:
an electromagnetic wave generation unit configured to generate a wave at a Schumann resonance frequency,
wherein the wave at the Schumann resonance frequency is applied to a cell.


SEQUENCE LISTING (7 sequences)

SEQ ID NO: 1 - RNA, 22 nt, Homo sapiens
  misc_feature: hsa-miR-181a-2-3p
accacugaccguugacuguacc

SEQ ID NO: 2 - RNA, 20 nt, Homo sapiens
  misc_feature: hsa-miR-181b-2-3p
cucacugaucaaugaaugca

SEQ ID NO: 3 - RNA, 22 nt, Homo sapiens
  misc_feature: hsa-miR-132-3p
uaacagucuacagccauggucg

SEQ ID NO: 4 - RNA, 21 nt, Homo sapiens
  misc_feature: hsa-miR-647
guggcugcacucacuuccuuc

SEQ ID NO: 5 - RNA, 19 nt, Homo sapiens
  misc_feature: hsa-miR-1973
accgugcaaagguagcaua

SEQ ID NO: 6 - RNA, 23 nt, Homo sapiens
  misc_feature: hsa-miR-145-5p
guccaguuuucccaggaaucccu

SEQ ID NO: 7 - RNA, 22 nt, Homo sapiens
  misc_feature: hsa-miR-4654
ugugggaucuggaggcaucugg